(19) United States
(12) Patent Application Publication
(10) Pub. No.: US 2025/0135181 A1
(43) Pub. Date: May 1, 2025

(54) CATHETER POSITIONING SYSTEM WITH SENSORY FEEDBACK

(71) Applicant: Abiomed, Inc., Danvers, MA (US)

(72) Inventors: Ali Almedhychy, Danvers, MA (US); Muhammad K. Sami, Danvers, MA (US); Christian Moyer, Danvers, MA (US)

(73) Assignee: Abiomed, Inc., Danvers, MA (US)

(21) Appl. No.: 18/910,424

(22) Filed: Oct. 9, 2024

Related U.S. Application Data
(60) Provisional application No. 63/589,400, filed on Oct. 11, 2023.

(51) Int. Cl. A61M 60/531; A61M 60/165; A61M 60/585; A61M 60/90

(52) U.S. Cl. CPC A61M 60/531; A61M 60/165; A61M 60/585; A61M 60/90; A61M 2205/04; A61M 2205/18; A61M 2205/3303; A61M 2205/3561; A61M 2205/3592; A61M 2205/502; A61M 2205/582; A61M 2210/125; A61M 2230/005; A61M 2230/04; A61M 2230/30

(57) ABSTRACT

Methods and apparatus for providing sensory feedback to an operator of a mechanical circulatory support (MCS) device are provided. The method comprises monitoring, using a controller of the MCS device, one or more physiological signals associated with a heart of a patient within which the MCS device is placed, generating an alert signal based, at least in part, on the monitored one or more physiological signals, and transmitting in response to generating the alert signal, a control signal from the controller of the MCS to a feedback device, wherein the feedback device is configured to provide sensory feedback to an operator of the MCS device based on the control signal.

### RELATED APPLICATIONS

This application claims the benefit under 35 U.S.C. § 119 (e) to U.S. Provisional Patent Application No. 63/589,400, filed Oct. 11, 2023 titled, “CATHETER POSITIONING SYSTEM WITH SENSORY FEEDBACK,” the entire contents of which is incorporated by reference herein.

### FIELD OF THE INVENTION

This disclosure relates to a catheter positioning system with sensory feedback.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span a heart valve. The intravascular blood pump is typically disposed at the end of the catheter. Once in position, the pump may be used to assist the heart and pump blood through the circulatory system and, therefore, temporarily reduce workload on the patient's heart, such as to enable the heart to recover after a heart attack. An exemplary intravascular blood pump is available from ABIOMED, Inc., Danvers, MA under the tradename Impella® heart pump.

Such pumps can be positioned, for example, in a cardiac chamber, such as the left ventricle, to assist the heart. In this case, the blood pump may be inserted via a femoral artery by means of a hollow catheter and introduced up to and into the left ventricle of a patient's heart. From this position, the blood pump inlet draws in blood and the blood pump outlet expels the blood into the aorta. In this manner, the heart's function may be replaced or at least assisted by operation of the pump.

An intravascular blood pump is typically connected to a respective heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller™. The controller raises alarms when operational data values fall beyond predetermined values or ranges, for example if a leak, suction, and/or pump malfunction is detected. The controller may include a video display screen upon which is displayed a graphical user interface configured to display the operational data and/or alarms.

### SUMMARY

Described herein are systems and methods for providing sensory feedback to an operator of a mechanical circulatory support (MCS) device, such as an intravascular blood pump. Initial placement of an MCS device into the heart of a patient is typically accomplished with the aid of imaging (e.g., fluoroscopic imaging, ultrasound imaging, etc.) to ensure that the pump is properly positioned. For instance, an operator (e.g., an interventionalist) may manipulate a catheter within which the blood pump is located to position a left-side MCS device such that the inlet of the blood pump is located in the left ventricle of the heart and the outlet of the blood pump is located in the aorta such that the pumping action of the blood pump spans the aortic valve. Following initial placement of the MCS device into a desired position, the MCS device may unintentionally become displaced due to various factors, examples of which are described herein. If the catheter is not manipulated to reposition the blood pump into the desired position, the patient may not be supported sufficiently by the MCS device. In some instances, the blood pump may act to destroy blood cells if, for example, both the inlet and the outlet of the blood pump are located in the ventricle while the pump is operating. Due to various factors such as other procedures (e.g., performing angioplasty) that they may be focused on, the operator of the MCS device may not immediately be aware that the device has deviated from its desired position and should be repositioned. Additionally, if the operator no longer has access to imaging (e.g., if the patient has been moved to the intensive care unit), it may be challenging to know during repositioning when the MCS device is back in a desired position. To this end, some embodiments of the present disclosure relate to systems and methods for providing the operator of an MCS device with sensory feedback to facilitate an operator's manipulation of a catheter to achieve proper placement of the device within a patient's heart.

Some embodiments relate to synchronizing visual and audible feedback with hemodynamic information separately from tactile feedback provided by a sensory feedback device. Some embodiments relate to providing sensory feedback (e.g., visual, audible, tactile feedback) timed with respect to a cardiac cycle of a patient. Some embodiments relate to providing sensory feedback to a dominant hand (or a hand that pushes the catheter) of an operator of an MCS device. Some embodiments relate to a sensory feedback system for guiding an operator to position an MCS device across the aortic valve of a patient without the use of a wire. Some embodiments relate to providing sensory feedback based on one or more push/pull characteristics (e.g., rate of push/pull) of a catheter manipulation. Some embodiments relate to providing sensory feedback in response to detecting a hazardous situation that may be impact device function or integrity.

In some embodiments, a method of providing sensory feedback to an operator of a mechanical circulatory support (MCS) device is provided. The method includes monitoring, using a controller of the MCS device, one or more physiological signals associated with a heart of a patient within which the MCS device is placed, generating an alert signal based, at least in part, on the monitored one or more physiological signals, and transmitting in response to generating the alert signal, a control signal from the controller of the MCS to a feedback device, wherein the feedback device is configured to provide sensory feedback to an operator of the MCS device based on the control signal.

In one aspect, the one or more physiological signals include an electrocardiogram signal. In another aspect, the one or more physiological signals include a pressure signal sensed by at least one pressure sensor associated with the MCS device. In another aspect, the pressure signal includes one or more of a ventricular pressure signal, an aortic pressure signal, or a differential pressure signal across at least one valve in the heart of the patient. In another aspect, generating the alert signal comprises generating the alert signal when it is determined based, at least in part, on the one or more physiological signals that the heart of the patient is in a systolic phase.

In another aspect, the sensory feedback includes tactile feedback. In another aspect, the tactile feedback includes vibratory feedback. In another aspect, the sensory feedback includes auditory feedback. In another aspect, the feedback device is a wearable device configured to be worn by the operator of the MCS device. In another aspect, the wearable device is configured to be worn on a wrist of the operator of the MCS device. In another aspect, the wearable device is configured to be worn on a finger of the operator of the MCS device. In another aspect, the wearable device is configured to be worn in an ear of the operator of the MCS device. In another aspect, the feedback device is coupled to a catheter associated with the MCS device.

In another aspect, transmitting the control signal comprises wirelessly transmitting the control signal from the controller of the MCS device to the feedback device. In another aspect, generating the alert signal comprises generating the alert signal when it is determined based, at least in part, on the one or more physiological signals, that the MCS device is not in a desired position within the heart of the patient. In another aspect, determining that the MCS device is not in a desired position comprises determining that an outlet of the MCS device is in a ventricle of the heart of the patient. In another aspect, generating the alert signal comprises generating the alert signal when it is determined based, at least in part, on the one or more physiological signals, that the MCS device is in a desired position within the heart of the patient. In another aspect, determining that the MCS device is in a desired position comprises determining that the MCS device is positioned across a valve in the heart of the patient. In another aspect, monitoring one or more physiological signals associated with a heart of a patient within which the MCS device is placed is performed during insertion of the MCS device into the heart of the patient.

In another aspect, the method further includes determining that the MCS device is being repositioned in the heart of the patient without reducing a pump speed of the MCS device, and generating an alert signal is further based, at least in part, on the determination that the MCS device is being repositioned in the heart of the patient without reducing the pump speed. In another aspect, determining that the MCS device is being repositioned in the heart of the patient without reducing a pump speed of the MCS device comprises detecting a femoral arterial pulse while the MCS device is operating. In another aspect, the method further includes receiving information associated with manipulation of the MCS device by the operator of the MCS device, and generating the alert signal is further based, at least in part, on the information associated with manipulation of the MCS device. In another aspect, the method further includes training a machine learning (ML) model based on the information associated with manipulation of the MCS device to generate a trained ML model, and generating the alert signal is further based, at least in part, on an output of the trained ML model.

In some embodiments, a mechanical circulatory support (MCS) device is provided. The MCS device includes a pump configured to be placed in a heart of a patient, a feedback device configured to provide sensory feedback to an operator of the MCS device, and a controller. The controller is configured to monitor one or more physiological signals associated with the heart of the patient during operation of the pump, generate an alert signal based, at least in part, on the monitored one or more physiological signals, and transmit in response to generating the alert signal, a control signal from the controller of the MCS to the feedback device. The feedback device is configured to provide the sensory feedback based on the control signal.

In one aspect, the one or more physiological signals include an electrocardiogram signal. In another aspect, the MCS device further includes at least one pressure sensor configured sense a pressure signal, and the one or more physiological signals include the pressure signal. In another aspect, the pressure signal includes one or more of a ventricular pressure signal, an aortic pressure signal, or a differential pressure signal across at least one valve in the heart of the patient. In another aspect, generating the alert signal comprises generating the alert signal when it is determined based, at least in part, on the one or more physiological signals that the heart of the patient is in a systolic phase.

In another aspect, the sensory feedback includes tactile feedback. In another aspect, the tactile feedback includes vibratory feedback. In another aspect, the sensory feedback includes auditory feedback. In another aspect, the feedback device is a wearable device configured to be worn by the operator. In another aspect, the wearable device is configured to be worn on a wrist of the operator. In another aspect, the wearable device is configured to be worn on a finger of the operator. In another aspect, the wearable device is configured to be worn in an ear of the operator.

In another aspect, the MCS device further includes a catheter, wherein the feedback device is coupled to the catheter. In another aspect, transmitting the control signal comprises wirelessly transmitting the control signal from the controller to the feedback device. In another aspect, generating the alert signal comprises generating the alert signal when it is determined based, at least in part, on the one or more physiological signals, that the pump is not in a desired position within the heart of the patient. In another aspect, determining that the pump is not in a desired position comprises determining that an outlet of the pump is in a ventricle of the heart of the patient. In another aspect, generating the alert signal comprises generating the alert signal when it is determined based, at least in part, on the one or more physiological signals, that the pump is in a desired position within the heart of the patient. In another aspect, determining that the pump is in a desired position comprises determining that the pump is positioned across a valve in the heart of the patient.

In another aspect, the controller is further configured to determine that the pump is being repositioned in the heart of the patient without reducing a pump speed of the pump, and generating an alert signal is further based, at least in part, on the determination that the pump is being repositioned in the heart of the patient without reducing the pump speed. In another aspect, determining that the pump is being repositioned in the heart of the patient without reducing a pump speed of the pump comprises detecting a femoral arterial pulse while the pump is operating. In another aspect, the controller is further configured to receive information associated with manipulation of the pump by the operator, an generating the alert signal is further based, at least in part, on the information associated with manipulation of the pump. In another aspect, the controller is further configured to train a machine learning (ML) model based on the information associated with manipulation of the pump to generate a trained ML model, and generating the alert signal is further based, at least in part, on an output of the trained ML model.

In some embodiments, a controller for a mechanical circulatory support (MCS) device is provided. The controller includes at least one hardware computer processor. The at least one hardware computer processor is programmed to generate an alert signal based, at least in part, on one or more physiological signals associated with a heart of a patient within which the MCS device is placed, and transmit, in response to generating the alert signal, a control signal to a feedback device, wherein the feedback device is configured to provide sensory feedback to an operator of the MCS device based on the control signal.

In some embodiments, a method of providing personalized feedback to an operator of an mechanical circulatory support (MCS) device is provided. The method includes receiving by a controller of the MCS device, information from one or more sensors, wherein the information is associated with manipulation of the MCS device by the operator of the MCS device, generating an alert signal based, at least in part, on the received information, and transmitting in response to generating the alert signal, a control signal from the controller of the MCS to a feedback device, wherein the feedback device is configured to provide sensory feedback to an operator of the MCS device based on the control signal.

In one aspect the method further includes training a machine learning (ML) model based on the information associated with manipulation of the MCS device to generate a trained ML model, and generating an alert signal is further based, at least in part, on an output of the trained ML model.

### BRIEF DESCRIPTION OF DRAWINGS

FIG. 1A illustrates a pump system, in accordance with some embodiments of the present disclosure.

FIG. 1B is a cross-sectional view of a portion of the pump system of FIG. 1A.

FIGS. 2A-2C illustrate examples of various sensory feedback devices, in accordance with some embodiments of the present disclosure.

FIG. 3 is a flowchart of a process for providing sensory feedback, in accordance with some embodiments of the present disclosure.

FIG. 4 illustrates a plurality of physiological signals used to detect a systolic pulse, in accordance with some embodiments of the present disclosure.

FIG. 5 schematically illustrates a system for providing sensory feedback to an operator based, at least in part, on monitored operator information, in accordance with some embodiments of the present disclosure.

FIG. 6 is a flowchart of a process for generating an alert signal based on an operator-specific model, in accordance with some embodiments of the present disclosure.

FIG. 7 schematically illustrates a system for providing sensory feedback to an operator of a mechanical circulatory support device, in accordance with some embodiments of the present disclosure.

### DETAILED DESCRIPTION

Transcatheter transvalvular pumps are increasingly adopted to provide hemodynamic support to patients with manifest or at the risk of reduced cardiac function. The transcatheter pump is typically inserted either through the femoral or axillary artery and introduced into the left ventricle through a transaortic retrograde route. Other heart pump types have been described to provide support to patients with right ventricle (RV) failure; in this scenario, the RV pump catheter is introduced either through the internal jugular or the femoral veins, through the central vasculature of the patient, and is finally positioned across heart valves spanning 2-3 heart chambers.

Pump insertion into the desired intra-cardiac position is typically achieved through a standard catheterization technique (e.g., access site might be either percutaneous or surgical), utilizing fluoroscopic imaging, and ultrasound imaging for position optimization.

Under certain disease conditions, patients may require prolonged support time with a mechanical cardiac support (MCS) device, such as a heart pump, beyond the time of the imaging guided procedure (e.g., percutaneous coronary intervention (PCI), surgery). In such instances, the patient is typically transferred, on-support, to the intensive care unit (ICU) or other units in the hospital facility where imaging support is not readily available. Under certain conditions of illness and/or prolonged support, the intra-cardiac pump may depart, unintentionally, from its originally-intended anatomical position in the patient's heart, thereby requiring repositioning to guarantee optimal support conditions. Such circumstances may arise from, for example, unintentional manipulation (push/pulls) of the extra-corporeal portion of the pump catheter causing the pump to exit its original position. Another situation could arise from development of acute decompensation of the left/right heart causing significant dilation of the affected chamber and loss of the catheter back-up support originally provided by cardiac structure prior to decompensation.

An operator who practices catheterization, including pump insertion is typically trained to receive information visually (from monitors, etc.) and to execute manipulation of the catheter manually based on visually received information. Despite being standard in cardiac catheterization, the above image-guidance is limited by the operator's (sensory followed by cortico-motoric) reaction time spanning the chain of receiving visual/auditory information, cortical processing and manual execution (typically in the form of holding, pushing or releasing the catheter). In certain clinical scenarios, this constitutes a critical delay especially when the operator is expected to execute manipulations of the catheter within milli-seconds time windows (e.g. Systolic phase of the cardiac cycle), during which the operator must notice the beginning of the systolic phase (e.g., from an EKG monitor), process and react in the form of catheter manipulation, prior to the end of the systolic phase.

The above scenario is one of several examples where an operator's reaction during catheterization must occur at ganglionic level and must bypass slower cortical processes. The following additional clinical scenarios can be presented, in which a swift imaging-free execution of the cascade of receiving physiological information, processing, and catheter execution would be desired:
Transcatheter crossing of the aortic valve, into the left ventricle (LV): Especially in the case of wireless introduction an intra-cardiac catheter; the crossing of the aortic valve (AV) is only possible during systolic phase (when the AV is physiologically open).
Repositioning of a transvalvular pump, originally located in intra-cardiac position, with the objective of optimizing pump position (e.g., into a transvalvular position).
Catheter manipulation in clinical situations where no imaging is available (e.g., bedside pump optimization).
Transcatheter intracoronary recanalization utilizing mechanical or laser means.
Cardiac electrophysiologic ablation.
Clinical scenario in which repositioning of an intra-cardiac pump is required, and pump operation must be reduced/minimized during the repositioning maneuver.

Some embodiments of the present disclosure relate to methods and apparatus for communicating physiological (e.g., hemodynamic) information to an extra-corporeal portion of a catheter by providing the operator with sensory feedback (e.g., tactile feedback) that can be detected by the operator while manipulating the extra-corporeal portion of catheter. As described in further detail below, in some embodiments, the sensory feedback may be provided on the catheter itself or via a device coupled to the catheter. In other embodiments, the sensory feedback may be provide on a wearable device (e.g., a bracelet, a watch, a ring, an in-ear insert) in wireless communication with a controller of the MCS device.

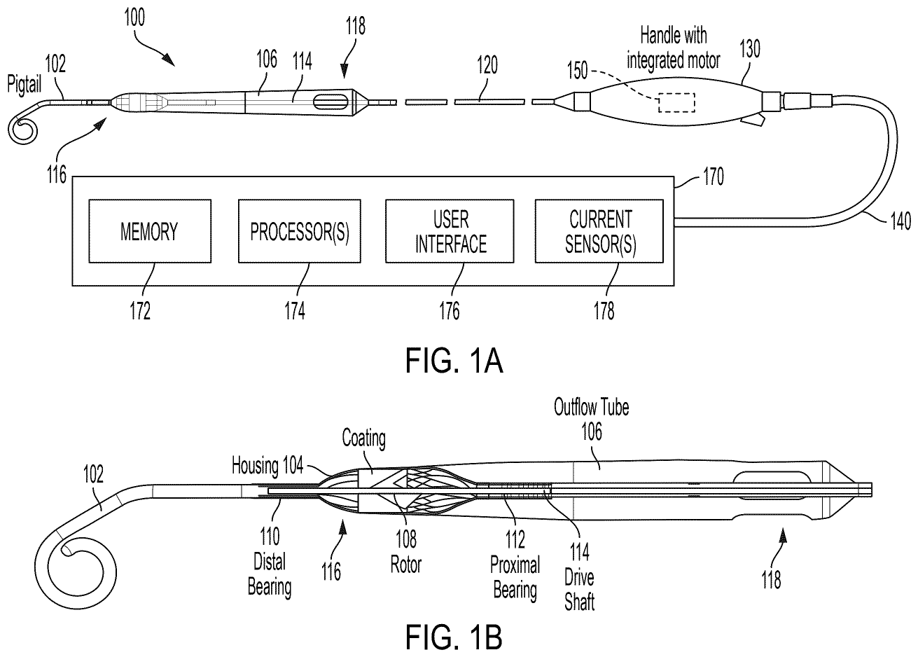

A pump system 100 for use with some embodiments of the present technology is shown in FIGS. 1A and 1B. As shown, pump system 100 is coupled to a control unit 200. Pump 100 includes a distal atraumatic tip 102, a pump housing 104 surrounding a rotor 108, an outflow tube 106, distal bearing 110, proximal bearing 112, inlet 116, outlet 118, catheter 120, handle 130, cable 140, and motor 150. Pump housing 104 may be configured as a frame structure formed by a mesh with openings which may, at least in part, be covered by an elastic material. A proximal portion of pump housing 104 extends into and is mounted in the hollow interior of outflow tube 106, and a distal portion of pump housing 104 extends distally beyond the distal end of outflow tube 106. The exposed openings in the pump housing 104 extending distally beyond outflow tube 106 form the inlet 116 of pump 100. The proximal end of outflow tube 106 includes a plurality of openings that form the outlet 118 of pump 100. Rotor 108 is rotationally mounted between distal bearing 110 and proximal bearing 112, and is coupled to a distal end of drive shaft 114. Drive shaft 114 is flexible and extends through catheter 120, through the hollow interior of outflow tube 106, into handle 130 and is coupled to motor 150, which is housed in handle 130. The proximal end of handle 130 is coupled via cable 140 to control unit 200. A fluid may be circulated through the catheter 120 proximate to the drive shaft 114 and in the space surrounding the distal bearing 110 and proximal bearing 112 to lubricate those components and reduce friction during operation of the pump 100.

Control unit 200 includes one or more memory 202, one or more processors 204, user interface 206, and one or more current sensors 208. Processor(s) 204 may comprise one or more microcontrollers, one or more microprocessors, one or more application specific integrated circuits (ASICs), one or more digital signal processors, program memory, or other computing components. Processor(s) 204 is communicatively coupled to the other components (e.g., memory 202, user interface 206, current sensor(s) 208) of control unit 200 and is configured to control one or more operations of pump 100. As a non-limiting example, control unit 200 may be implemented as an Automated Impella Controller™ from ABIOMED, Inc., Danvers, MA. In some aspects, memory 202 is included as a portion of processor(s) 204 rather than being provided as a separate component.

During operation, processor(s) 204 is configured to control the electrical power delivered to motor 150 (e.g., by controlling a power supply (not shown)) by a power supply line (not shown) in cable 140, thereby controlling the speed of the motor 150. Current sensor(s) 208 may be configured to sense motor current associated with an operating state of the motor 150, and processor(s) 204 may be configured to receive the output of current sensor(s) 208 as a motor current signal. Processor(s) 204 may further be configured to determine a flow through the pump 100 based, at least in part, on the motor current signal and the motor speed, as described in more detail below. Current sensor(s) 208 may be included in control unit 200 or may be located along any portion of the power supply line in cable 140. Additionally or alternatively, current sensor(s) 208 may be included in motor 150 and processor(s) 204 may be configured to receive the motor current signal via a data line (not shown) in cable 140 coupled to processor(s) 204 and motor 150.

Memory 202 may be configured to store computer-readable instructions and other information for various functions of the components of control unit 200. In one aspect, memory 202 includes volatile and/or non-volatile memory, such as, an electrically erasable programmable read-only memory (EEPROM).

User interface 206 may be configured to receive user input via one or more buttons, switches, knobs, etc. Additionally, user interface 206 may include a display configured to display information and one or more indicators, such as light indicators, audio indicators, etc., for conveying information and/or providing alerts regarding the operation of pump 100.

Pump 100 is designed to be insertable into a patient's body, e.g., into a left ventricle of the heart, with an introducer system. In one aspect, housing 104, rotor 108, and outflow tube 106 are radially compressible to enable pump 100 to achieve a relatively small outer diameter of, for example, 9 Fr (3 mm) during insertion. When pump 100 is inserted into the patient, e.g., into a left ventricle, handle 130 and motor 150 remain disposed outside the patient. It should be appreciated that in other embodiments, motor 150 may be inserted into the patient (e.g., by being located onboard the pump 100). During operation, motor 150 is controlled by processor(s) 204 to drive rotation of drive shaft 114 and rotor 108 to convey blood from inlet 116 to outlet 118. It is to be appreciated that rotor 108 may be rotated by motor 150 in reverse to convey blood in the opposite direction (in this case, the openings of 118 form the inlet and the openings of 116 form the outlet). In one aspect, pump 100 is intended to be used during high-risk procedures for a duration of up to six hours, up to one day, up to one week, up to two weeks, up to one month, etc., though it should be understood that the technology described herein is not limited to any particular types of procedures and/or use durations.

As described above, some embodiments of the present disclosure relate to a system and methods for providing sensory feedback to an operator tasked with positioning a mechanical circulatory support (MCS) device (e.g., pump 100) within a heart of a patient by manipulating a catheter (e.g., catheter 120) associated with the pump. Such sensory feedback may facilitate initial placement or repositioning of the pump within the heart of the patient into a desired position (e.g., across the aortic valve for a left-side device). In some embodiments, sensory feedback may be provided to an operator of an MCS device to alert the operator to take certain actions, such as reducing the speed of the pump prior to repositioning and/or removing the pump from the heart of the patient. Sensory feedback may be provided for any other suitable reason, examples of which are discussed in further detail herein.

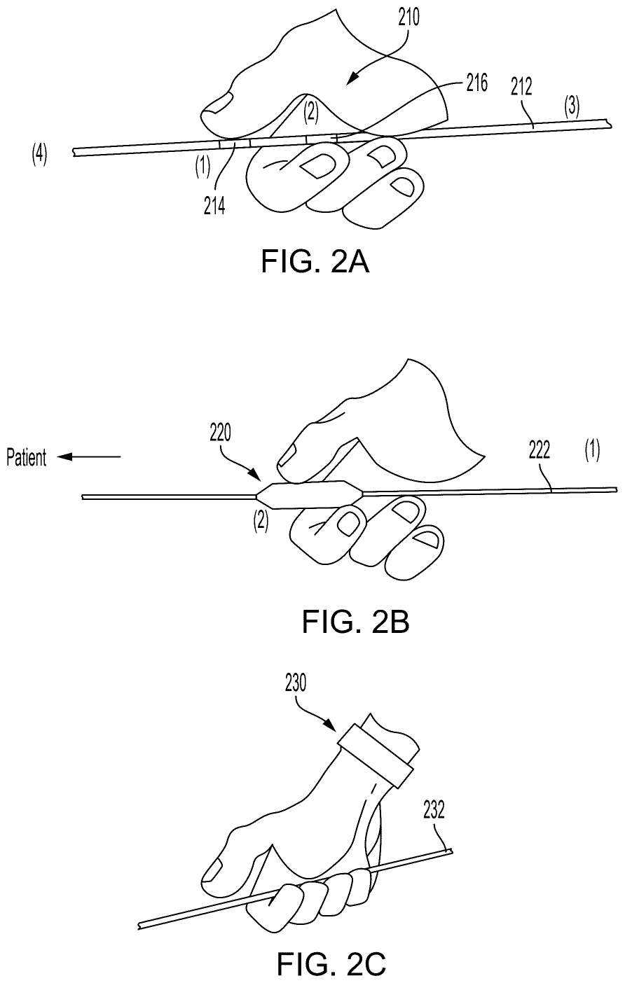

FIGS. 2A-2C schematically illustrate example feedback devices that may be used to provide sensory feedback to an operator of an MCS device in accordance with some embodiments of the present disclosure. FIG. 2A shows a first example of a feedback device 210 coupled to a catheter 212, in accordance with some embodiments. As shown in FIG. 2A, feedback device 210 includes a first sensor 214 and a second sensor 216, each of which is coupled to the shaft of catheter 212. The first sensor 214 may be arranged to engage with the thumb of the operator and the second sensor 216 may be arranged to engage with another finger (e.g., the index finger) of the operator when the operator manipulates the catheter 212. In the embodiment shown in FIG. 2A, the first sensor 214 and the second sensor 216 may be configured to provide tactile (e.g., vibratory) feedback to the operator as the operator manipulates the catheter 212 (e.g., during insertion or repositioning of an MCS device).

FIG. 2B shows a second example of a feedback device 220 coupled to a catheter 222, in accordance with some embodiments of the present disclosure. As shown in FIG. 2B, feedback device 220 is a vibration-enabled device configured to provide tactile feedback to an operator's hand during catheter manipulation (e.g., during insertion or repositioning of an MCS device). In some embodiments, feedback device 220 may be configured to reversibly couple to catheter 222 by, for example, snapping onto the shaft of catheter 222.

FIG. 2C shows a third example of a feedback device 230. Feedback device 230 is not directly coupled to catheter 232, but instead is implemented as a wearable device configured to be worn by an operator of an MCS device. As shown in FIG. 2C, feedback device 230 is a bracelet that can be worn about a wrist of the operator. It should be appreciated that when implemented as a wearable device, feedback device 230 may be implemented in any suitable way, examples of which include, but are not limited to, a ring to be worn around a finger of the operator, an in-ear device configured to be worn in an ear of the operator, an earring configured to clipped to or worn in an ear of the operator, a palm strap configured to be worn around a hand of the operator, or a necklace configured to be worn about the neck of the operator. In some embodiments, feedback device 230 may be implemented in or coupled to a wearable accessory that has functionality or purpose other than providing sensory feedback. For instance, feedback device 230 may be implemented or coupled to glasses, a smartwatch, or attached to a clip that can be clipped on scrubs or other clothing. Although the example feedback devices described herein are configured to provide sensory feedback in the form of tactile (e.g., vibratory) feedback, it should be appreciated that the sensory feedback may additionally or alternatively be provided as visual or auditory feedback. For instance, when implemented as an in-ear wearable device, the feedback device may be configured to provide one or more audible tones to alert the operator. Additionally, tactile sensory feedback may include, but is not limited to, light touch, discriminative touch, touch pressure, pain, temperature, or vibration.

A feedback device for providing sensory feedback to an operator of an MCS device in accordance with the techniques described herein may be configured to receive control signals from another device (e.g., a controller of the MCS device, a processor in communication with the controller of the MCS device). For instance, when implemented as a wearable device, the feedback device may include a wireless communication interface configured to wirelessly receive control signals from the controller of the MCS device and to provide sensory feedback to the operator in response to receiving the control signals. In some embodiments, the feedback device may be configured to provide sensory feedback to the operator based on a configuration of the feedback device and/or operator preferences stored in an operator profile.

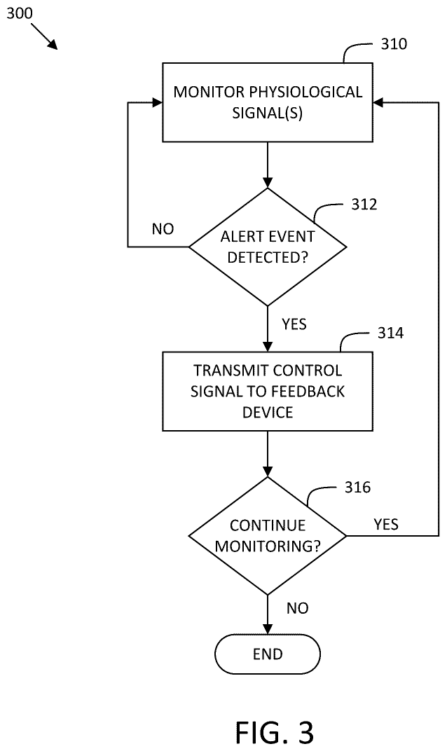

FIG. 3 illustrates a process 300 for providing sensory feedback to an operator of an MCS device, in accordance with some embodiments of the present disclosure. Process 300 may begin in act 310, where one or more physiological signals are monitored. The MCS device may include one or more sensors (e.g., pressure sensors) configured to sense physiological signals (e.g., aortic pressure, left ventricular pressure, etc.), which may be monitored. Process 300 may then proceed to act 312, where the monitored physiological signal(s) are processed to determine whether an alert event is detected. For instance, a controller of the MCS device (or one or more processors in communication with the controller of the MCS device) may receive the sensed physiological signals from the sensor(s) of the MCS device and may process the signals to detect one or more alert events. In some embodiments, the physiological signal(s) may be received from multiple devices associated with sensors configured to sense the physiological signals. In some embodiments, the physiological signals may include, but are not limited to, hemodynamic signals, EKG signals, pressure tracking signals, MCS position detecting signals, or one or more derived signals.

In some embodiments, an alert event may represent that the blood pump is not in a desired position within the heart of the patient. For example, a desired position of a left-side MCS device may be such that the inlet of the pump resides in the left ventricle and the outlet of the pump resides in the aorta such that blood is pumped through the aortic valve. Some MCS devices may include one or more pressure sensors configured to sense the pressure at the inlet, the outlet, or both the inlet and the outlet of the MCS device. Pressure signals sensed by the pressure sensor(s) may be used to determine the position of the MCS within the patient's heart.

For instance, when the pump is located entirely within the left ventricle, the differential pressure between the inlet and outlet may be zero (or close to zero) indicating that the pump is not properly positioned across the aortic valve. When such a situation occurs, an alert event may be detected in act 312, and a control signal may be transmitted to the feedback device in act 314 to provide sensory feedback to the operator. In some embodiments, the control signal transmitted to the feedback device may be the same regardless of the type of alert event detected. In other embodiments, information about the type of alert event may be coded in the control signal transmitted to the feedback device. By coding alert event type information into the control signal, the feedback device may provide different types of sensory feedback for different types of detected alert events. For instance, the different types of sensory feedback may include different vibratory stimulus sequences and/or intensities, different modalities of sensory feedback or combinations thereof.

In some embodiments, detecting that the MCS device is not in a desired position may include one or more of the following scenarios—the catheter is fully in the ventricle, the catheter is in close proximity to ventricular structure, including mitral valve leaflets, cordae, or papillary muscles, the catheter is in close proximity to aortic valve structure, including aortic valve leaflets, the catheter is fully in the aorta, or the catheter is fully in arterial tree

It should be appreciated that detecting that the pump is not in a desired position is merely one example of an alert event that can be detected using one or more of the techniques described herein. For instance, an alert event may be detected when, during manipulation of the catheter, the pump is detected to be in a desired position to inform the operator to cease manipulation of the catheter. In other instances, an alert event may be detected when it is determined that the catheter is being withdrawn from the heart of the patient, but that the pump speed has not been reduced. As such, it should be appreciated that in addition to physiological signals associated with the patient, one or more signals (e.g., pump speed, motor current, etc.) associated with operation of the MCS device may in some embodiments, be taken into consideration when determining in act 312 whether an alert event is detected.

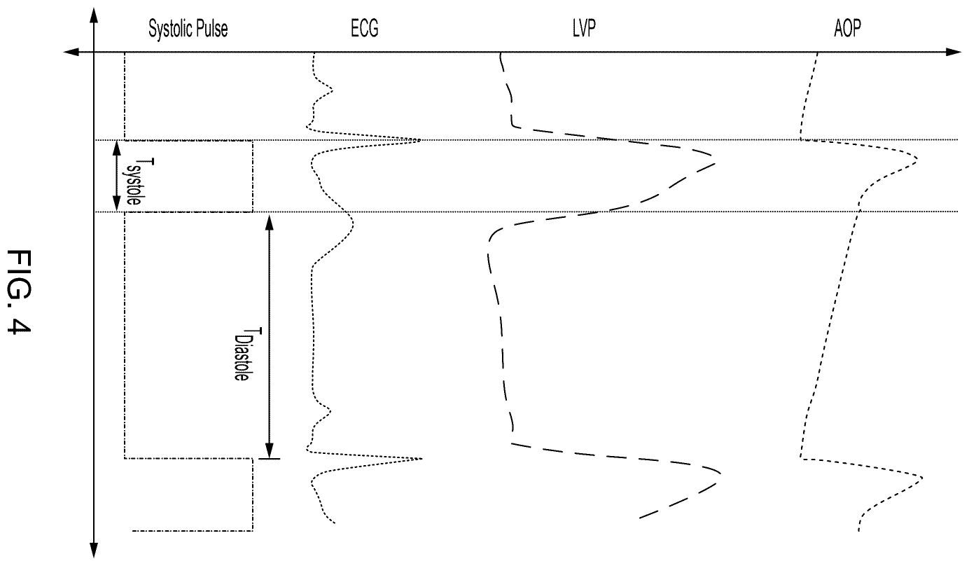

In some embodiments, the inventors have recognized that it may be useful to identify to the operator during manipulation of the catheter, a timing of the systolic phase of the patient's cardiac cycle. To properly position a left side MCS device across the aortic valve, the pump must traverse the aortic valve when it is open (during systole). Using conventional techniques, the operator may rely on trial and error to advance the pump past the aortic valve. Alternatively, the operator may view the EKG waveform and/or one or more pressure signals displayed on a display in an attempt to identify the start of the systolic phase of the patient's cardiac cycle. However, due to the time needed for the operator's brain to process the displayed signals (e.g., using cortical processing), it is often challenging for the operator to react in sufficient time to advance the pump during the relatively short systolic phase. Some embodiments of the present disclosure process one or more physiological signals for a patient to detect the beginning of the systolic phase of the patient's cardiac cycle as an alert event. In such instances, sensory feedback may be provided to the operator with considerably shorter lag time compared to the conventional visual processing techniques described above. FIG. 4 illustrates an example of how one or more physiological signals can be used to detect the onset of the systolic phase of a cardiac cycle, in accordance with some embodiments. As shown in FIG. 4, the beginning of a systolic pulse (having a duration T_{Systole}) may be detected based on the peak in the electrocardiogram (ECG) signal, with the peaks in left ventricular pressure (LVP) and aortic pressure (AOP) also occurring within the systolic pulse. By monitoring one or more of these physiological signals, the systolic pulse as an alert event may be reliably detected, such that sensory feedback may be provided to an operator of an MCS device during catheter manipulation. The sensory feedback may enable the operator to advance the MCS device though the aortic valve in a more informed manner than using conventional approaches, which may reduce the amount of time needed to place the pump in the proper position in the heart of a patient.

Returning to process 300 shown in FIG. 3, if no alert event is detected in act 312, process 300 may return to act 310, where the one or more physiological signals continue to be monitored. When an alert event is detected in act 312, process 300 may proceed to act 314, where a control signal is transmitted to a feedback device, wherein the feedback device is configured to provide sensory feedback to an operator of the MCS device, as described above. In some embodiments, the control signal is wirelessly transmitted to the feedback device, which may be coupled to the catheter or arranged on a wearable device, examples of which are described herein. Process 300 may then proceed to act 316, where it is determined whether to continue monitoring for alert events. If it is determined in act 316 to continue monitoring for alert events, process 300 may return to act 310 where the one or more physiological signals are monitored. If it is determined in act 316 that monitoring for alert events is no longer desired, process 300 may end.

The inventors have recognized and appreciated that there is variability between operators in how they perform catheter manipulation, and that it may be helpful to train a model (e.g., a machine learning (ML) model) such that the model is tuned to the behavior of an individual operator to provide personalized predictions regarding alert events. The output of the trained model may then be used to adjust the timing of providing the sensory feedback to a particular operator, which may further facilitate the operator's understanding of how to manipulate the catheter. For instance, continuing with the example of analyzing one or more physiological signals to detect the onset of the systolic phase of a patient's cardiac cycle, a timing of transmitting a control signal to the feedback device may be adjusted based, at least in part, on an operator's behavior (e.g., reaction time) when receiving the sensory feedback. In this way, the system may learn how different operators perform catheter manipulation, which may in turn be used to facilitate catheter manipulation operations to achieve proper placement of an MCS device in a patient.

In some embodiments the timing of providing the sensory feedback may adjusted based on other factors. For instance, the timing of providing the sensory feedback may be adjusted based on data acquisition delays introduced by a patient device or monitor, which provides the physiological signals for processing. As another example, the timing of providing the sensory feedback may be adjusted based on data communication delays introduced by wired or wireless communications in various components of the data communication system and/or data processing delays introduced by one or more data processing components. As yet further examples, the timing of providing the sensory feedback may be adjusted based on one or more of a Visual Reaction Time (VRT) of the operator when visual sense is used to perform the task (e.g., catheter manipulation), Audible Reaction Time (ART) of the operator when audio sense is used to perform the task, Tactile Reaction Time (TRT) of the operator when tactile sense is used to perform the task, or a combination VRT, ART, and TRT when a combination of Visual, Audible and Tactile senses are used to perform the task.

In some embodiments of the present disclosure, sensory feedback may cease to be provided when the sensory feedback system enters a particular mode. For example, the system may stop providing the operator with systolic pulse stimulation when one or more of the following takes place: the pump is detected to be in the correct position, arrythmia is detected, a suction condition is detected, or a clot ingestion signature is detected. In some embodiments, sensory feedback associated with systolic phase detection may be initiated in response to an operator-initiated request and/or the pump is detected to be in the aorta.

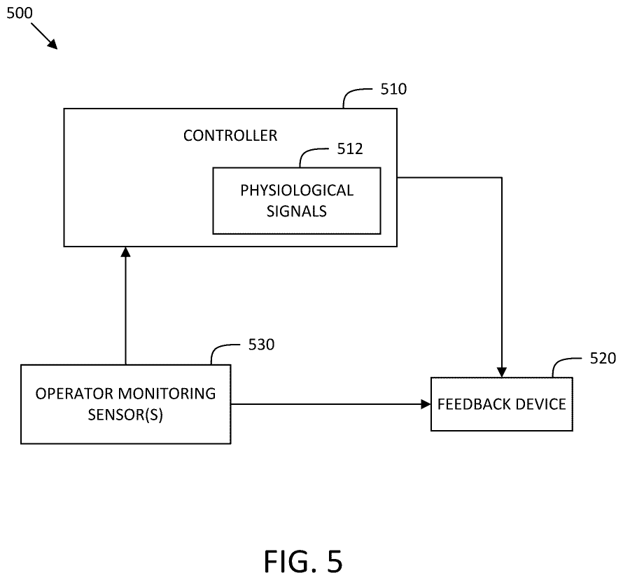

FIG. 5 illustrates a system 500 for providing operator-specific tuning of sensory feedback to an operator of an MCS device, in accordance with some embodiments of the present disclosure. As shown in FIG. 5, system 500 includes controller 510 (e.g., a controller of an MCS device). As described herein, controller 510 may be configured to process one or more physiological signals 512 sensed by one or more sensors associated with the MCS device (e.g., AOP, LVP) or sensed using another device (e.g., ECG signal). Based on monitoring the physiological signal(s) 512 for an alert event, controller 510 may be configured to transmit a control signal to feedback device 520 to provide sensory feedback to an operator of the MCS device (e.g., an operator performing catheter manipulation). System 500 also includes one or more operator monitoring sensors 530 configured to monitor the operator's movements during catheter manipulation. For instance, the feedback device 520 may include one or more sensors (e.g., gyroscopes) that sense the movements of the hand of the operator. Other examples of operator monitoring sensors 530 include, but are not limited to, motion sensors, EMG sensors associated with the operator's hand, or spatial computing inputs (e.g., cameras capturing images of the operator's hands). The information about the operator's hand movements may be used to train an operator-specific model, which controller 510 (or some other processing component) may use to adjust the timing of the sensory feedback provided by feedback device 520. In some embodiments, the adjustment of the sensory feedback may be provided by the feedback device 520 itself. For instance, each operator may be associated with an operator-specific feedback device that has been tuned to their particular movement characteristics, and may operate to provide sensory feedback accordingly.

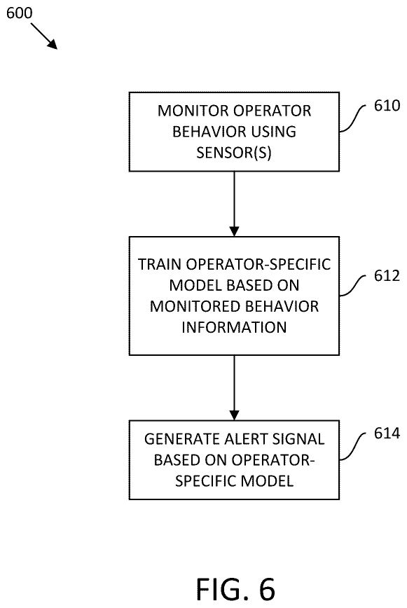

FIG. 6 is a flowchart of a process 600 for providing sensory feedback based on an operator-specific model, in accordance with some embodiments. Process 600 begins in act 610, where operator behavior is monitored using one or more sensors. For example, as described above, one or more operator monitoring sensors may be used to detect the movements of an operator's hand as they manipulate a catheter associated with an MCS device. Process 600 then proceeds to act 612, where the operator behavior information sensed by the one or more sensors is used to train an operator-specific model. For example, the operator-specific model may be a machine learning model, and the machine learning model may be trained based on the sensor information to represent characteristics of the operator's behavior that may be used to tailor the timing of how sensory information is provided to the operator using a feedback device. Process 600 then proceeds to act 614, where the trained operator-specific model is used to generate an alert signal upon which sensory feedback may be based, as described herein. In this way, some embodiments provide sensory feedback that is individualized to a particular operator based on their behavior profile as captured in the trained operator-specific model.

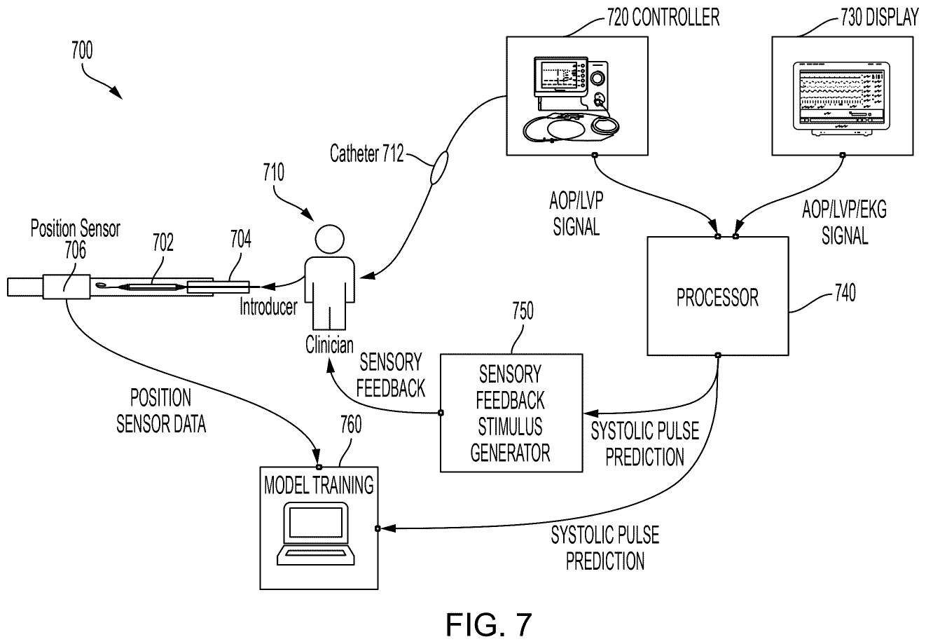

FIG. 7 schematically illustrates components of a sensory feedback system 700, in accordance with some embodiments of the present disclosure. In the example shown in FIG. 7, sensory feedback system 700 is configured to process physiological signals to detect an alert event as the onset of the systolic phase of a patient's cardiac cycle, and to provide sensory feedback to an operator based on the detection. It should be appreciated, however, that the sensory feedback system 700 may be configured to detect other types of alert events, non-limiting examples of which are described herein.

Sensory feedback system 700 includes controller 720 configured to control a heart pump 702 coupled thereto. An operator 710 may insert the heart pump 702 into the heart of a patient using a catheter 712 and a catheter introducer 704. An example of inserting a heart pump into the heart of a patient is described herein in connection with FIGS. 1A and 1B. Controller 720, heart pump 702 and catheter 712 may collectively form a mechanical circulatory support (MCS) device configured to support the cardiac functioning of the patient within which the heart pump 702 is placed. As described herein, the operator 710 may manipulate the catheter 712 to advance the heart pump 702 along a desired path and into the patient's heart. When properly positioned, the heart pump 702 may have its inlet located in the left ventricle and its outlet in the aorta of the patient's heart. To achieve such a position the heart pump 702 must pass through the aortic valve when open (i.e., during systole). Sensory feedback system 700 may include a feedback device (not shown) configured to provide the operator 710 with sensory feedback that alerts the operator when the aortic valve is open and the heart pump 702 can be advanced into the desired position across the valve.

As shown in FIG. 7, sensory feedback system 700 includes a processor 740 configured to receive one or more physiological signals related to the patient. For instance, processor 740 may receive one or more measured or derived pressure signals (e.g., left ventricular pressure (LVP), aortic pressure (AOP)) from controller 720 and/or one or more other signals (e.g., EKG signal) from display 730, which may be configured to display one or more waveforms or values based on information from controller 720 or other devices associated with the patient. Processor 740 is shown as a device separate from controller 720, though it should be appreciated in some embodiments, that processor 740 may be included as a portion of controller 720 or display 730, and embodiments of the present disclosure are not limited in this respect. Processor 740 may be configured to process the received physiological signals to detect or predict the occurrence of an alert event, such as the onset of a systolic pulse. Non-limiting examples of techniques for predicting the onset of a systolic pulse based on one or more physiological signals are described herein. For instance, in some embodiments, processor 740 may be configured to process the one or more physiological signals using a model (e.g., a machine learning model), with the output of the model being a prediction of a systolic pulse (or any other suitable alert event). Information about the systolic pulse prediction output from the processor 740 may be sent to sensory feedback stimulus generator 750. For example, processor 740 may send a control signal to sensory feedback stimulus generator 750, which may be implemented as a controller for a feedback device that provides sensory feedback to the operator 710. Processor 740 may also send the systolic pulse prediction to model training computing system 760. Model training computing system 760 may be configured to train a model used by processor 740 for predicting the systolic pulse, and in addition to receiving the systolic pulse prediction from processor 740 may also receive position sensor data from a position sensor 706 associated with the heart pump 702 and/or other information (e.g., operator movement information) that may be used to train an operator-specific model for predicting the systolic pulse.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of providing sensory feedback to an operator of a mechanical circulatory support (MCS) device, the method comprising:
monitoring, using a controller of the MCS device, one or more physiological signals associated with a heart of a patient within which the MCS device is placed;
generating an alert signal based, at least in part, on the monitored one or more physiological signals; and
transmitting in response to generating the alert signal, a control signal from the controller of the MCS to a feedback device, wherein the feedback device is configured to provide sensory feedback to an operator of the MCS device based on the control signal.

2. The method of claim 1, wherein the one or more physiological signals include an electrocardiogram signal and/or a pressure signal sensed by at least one pressure sensor associated with the MCS device.

3. (canceled)

4. (canceled)

5. The method of claim 1, wherein generating the alert signal comprises generating the alert signal when it is determined based, at least in part, on the one or more physiological signals that the heart of the patient is in a systolic phase.

6. The method of claim 1, wherein the sensory feedback includes tactile feedback.

7. (canceled)

8. (canceled)

9. The method of claim 1, wherein the feedback device is a wearable device configured to be worn by the operator of the MCS device.

10-12. (canceled)

13. The method of claim 1, wherein the feedback device is coupled to a catheter associated with the MCS device.

14. The method of claim 1, wherein transmitting the control signal comprises wirelessly transmitting the control signal from the controller of the MCS device to the feedback device.

15. The method of claim 1, wherein generating the alert signal comprises generating the alert signal when it is determined based, at least in part, on the one or more physiological signals, that the MCS device is not in a desired position within the heart of the patient.

16. (canceled)

17. The method of claim 1, wherein generating the alert signal comprises generating the alert signal when it is determined based, at least in part, on the one or more physiological signals, that the MCS device is in a desired position within the heart of the patient.

18. (canceled)

19. The method of claim 1, wherein monitoring one or more physiological signals associated with a heart of a patient within which the MCS device is placed is performed during insertion of the MCS device into the heart of the patient.

20. The method of claim 1, further comprising:
determining that the MCS device is being repositioned in the heart of the patient without reducing a pump speed of the MCS device, wherein
generating an alert signal is further based, at least in part, on the determination that the MCS device is being repositioned in the heart of the patient without reducing the pump speed.

21. The method of claim 20, where determining that the MCS device is being repositioned in the heart of the patient without reducing a pump speed of the MCS device comprises detecting a femoral arterial pulse while the MCS device is operating.

22. The method of claim 1, further comprising:
receiving information associated with manipulation of the MCS device by the operator of the MCS device, wherein
generating the alert signal is further based, at least in part, on the information associated with manipulation of the MCS device.

23. The method of claim 22, further comprising:
training a machine learning (ML) model based on the information associated with manipulation of the MCS device to generate a trained ML model, wherein
generating the alert signal is further based, at least in part, on an output of the trained ML model.

24. A mechanical circulatory support (MCS) device, comprising:
a pump configured to be placed in a heart of a patient;
a feedback device configured to provide sensory feedback to an operator of the MCS device; and
a controller configured to:
monitor one or more physiological signals associated with the heart of the patient during operation of the pump;
generate an alert signal based, at least in part, on the monitored one or more physiological signals; and
transmit in response to generating the alert signal, a control signal from the controller of the MCS to the feedback device,
wherein the feedback device is configured to provide the sensory feedback based on the control signal.

25-27. (canceled)

28. The MCS device of claim 24, wherein generating the alert signal comprises generating the alert signal when it is determined based, at least in part, on the one or more physiological signals that the heart of the patient is in a systolic phase.

29-35. (canceled)

36. The MCS device of claim 24, further comprising:
a catheter, wherein the feedback device is coupled to the catheter.

37-41. (canceled)

42. The MCS device of claim 24, wherein the controller is further configured to:
determine that the pump is being repositioned in the heart of the patient without reducing a pump speed of the pump, wherein
generating an alert signal is further based, at least in part, on the determination that the pump is being repositioned in the heart of the patient without reducing the pump speed.

43. (canceled)

44. The MCS device of claim 24, wherein the controller is further configured to:
receive information associated with manipulation of the pump by the operator, wherein
generating the alert signal is further based, at least in part, on the information associated with manipulation of the pump.

45. (canceled)

46. A controller for a mechanical circulatory support (MCS) device, the controller comprising:
at least one hardware computer processor programmed to:
generate an alert signal based, at least in part, on one or more physiological signals associated with a heart of a patient within which the MCS device is placed; and
transmit, in response to generating the alert signal, a control signal to a feedback device, wherein the feedback device is configured to provide sensory feedback to an operator of the MCS device based on the control signal.

47-48. (canceled)
